(19) United States
(12) Patent Application Publication
(10) Pub. No.: US 2015/0164692 A1
(43) Pub. Date: Jun. 18, 2015

(54) Method and system for endoscope-assisted non-invasive laser treatment of presbyopia

(71) Applicant: J.T. Lin, Brooklyn, NY (US)

(72) Inventor: J.T. Lin, Brooklyn, NY (US)

(21) Appl. No.: 13/998,858

(22) Filed: Dec. 16, 2013

(51) Int. Cl. A61F 9/008; A61B 1/07; A61B 1/05; A61B 1/00; A61B 18/24

(52) U.S. Cl. CPC A61F 9/00823; A61B 1/00087; A61B 18/24; A61B 1/05; A61B 1/07

(57) ABSTRACT

A method and system for treating presbyopia by an endoscope-assisted laser system consists of a camera connected to a signal-fiber, an illumination-fiber and a laser device connected to a laser-fiber. The endoscope gauge probe is inserted into the eye for real time monitoring of the photocoagulation process of the ciliary-body (or process) of the eye in a predetermined area inside the eye. The preferred laser device produces a laser beam having a wavelength 0.7 to 1.3 micron, laser fluency about 0.5 to 5.0 W/cm̂2, and operated at a continuous mode, preheating mode or a pulsed-mode having a repetition rate of (0.5-5,000) Hz. The presbyopia treatment can be combined with glaucoma or cataract procedure to achieve multi-function in single system.

### BACKGROUND OF THE INVENTION

1. Field of the Invention

The present invention relates to method and apparatus for ophthalmic treatments and, more particularly, for eye disorders such as presbyopia by laser thermal energy which changes the tissue property and structure of the ciliary body (process) to increase the accommodation of the eye, where the procedure is real time monitored by an endoscope device.

2. Description of Related Arts

Corneal reshaping including procedures of photorefractive keratectomy (PRK) and laser assisted in situ keratomileusis (LASIK) have been performed by lasers in the ultraviolet (UV) wavelength of (193-213) nm. The commercial UV refractive lasers include ArF excimer laser (at 193 nm) proposed by U.S. Pat. No. 4,773,414 of L'Esperance, et al. and other non-excimer, solid-state lasers such as those proposed by the present inventor in 1992 (U.S. Pat. No. 5,144,630) and in 1996 (U.S. Pat. No. 5,520,679). The above-described prior arts using lasers to reshape the corneal surface curvature, however, are limited to the corrections of myopia, hyperopia, monavision-presbyopia or astigmatism.

Refractive surgery using a scanning device and lasers in the mid-infrared (mid-IR) wavelength was first proposed by the present inventor in U.S. Pat. No. 5,144,630 and 5,520,679 and later proposed by Telfair et al. in U.S. Pat. No. 5,782,822, where the generation of mid-IR wavelength of (2.5-3.2) microns were disclosed by various methods including: the Er:YAG laser (at 2.94 microns), the Raman-shifted solid-state lasers (at 2.7-3.2 microns) and the optical parametric oscillation (OPO) lasers (at 2.7-3.2 microns).

All the above-described prior arts use methods to change the cornea surface curvature either by tissue ablation (such as in UV laser LASIK) or by thermal shrinkage (such as Ho:YAG laser and CK). In addition, the treated area is limited within the central potion of the cornea having a diameter about 6 to 8 mm. The above prior arts, therefore, did not actually resolve the intrinsic problems of presbyopia patient caused by age, where the decrease of accommodation as a result of the increase of the regality of the lens, ciliary-body or scleral layer.

The direct method for presbyopia correction, therefore, is to increase the accommodation of the presbyopic eyes by changing the intrinsic properties of the sclera or ciliary tissue without changing the cornea curvature. Because there is no reshaping of the cornea, the treated eye shall keep its original far vision while its near vision is improved after the presbyopia treatment. This is the fundamental difference between corneal reshaping and sclera-ciliary tissue ablation or coagulation.

To treat presbyopia patients using the concept of expanding the sclera by sclera expansion band (SEB) was proposed by Schachar in U.S. Pat. Nos. 5,489,299, 5,722,952, 5,465,737 and 5,354,331. The mechanical SEB approach has the drawbacks of complexity, major invasive, time consuming, potential side effects and postoperative regression.

Presbyopia correction using various lasers to remove a portion of the scleral tissue were proposed in many prior arts including U.S. Pat. Nos. 6,258,082, 6,263,879, 6,824,540 and 7,275,545 issued to the present inventor, and U.S. Pat Nos. 7,867,223, 8,276,593 and 8,479,745 issued to others. All of these prior arts require a laser being strongly absorbed by the sclera tissue which can be evaporated or ablated by the laser. Therefore these prior art s suffer the drawback of being an invasive surgical procedure with bleeding and potential of infection due to the exposure of the cut areas. In addition, the procedure is very slow, typically over 30 minutes each eye.

One objective of the present invention, therefore, is to provide an apparatus and method to obviate the drawbacks of the prior arts. In particular, a procedure which is non-invasive or minimally invasive, no bleeding, fast tissue healing, safer and no scleral tissue ablation or cutting. The proposed procedure of the present invention will be also 5 to 20 times faster than prior arts.

It is yet another objective of the present invention to provide a real time monitoring of the procedure by an endoscope device which consists of a gauge probe.

It is yet another objective of the present invention to provide new mechanisms based on laser interaction with the ciliary body, rather than ablation of the scleral tissue proposed by all prior arts. The present invention provides a non-surgical procedure which is also monitored by an endoscope. In addition, the proposed lasers in the present invention require wavelength in the near infrared (0.7-1.3 um) range, in contract to the mid-infrared wavelength (2.7-3.2 um) of the prior arts such as U.S. Pat. No. 6,258,082, U.S. Pat. No. 6,263,879, U.S. Pat. No. 6,824,540, U.S. Pat. No. 7,275,545, U.S. Pat. No. 7,867,223, U.S. Pat. No. 8,276,593 and U.S. Pat. No. 8,479,745.

Further objectives of the invention will become apparent from the description of the invention to be detailed as follows.

### SUMMARY OF THE INVENTION

A method for treating presbyopia comprising an endoscope-assisted laser system which consists of a camera connected to a signal fiber, a white light generator connected to an illumination fiber and a laser device connected to a laser fiber. These three fibers are guided to the treating area by an endoscope hand piece having a gauge probe. The laser device produces a laser beam having a wavelength between 0.7 and 1.3 micron. The endoscope gauge probe is inserted into the eye for real time monitoring of the photocoagulation process. The thermal energy of the laser beam is delivered to the soft tissue including ciliary-body (or process) of the eye in a predetermined area in the anterior chamber of the eye. The said soft tissue absorbing the laser thermal energy can affect at least one property of the eye and enhance an accommodation of the eye. The laser beam thermal effects include at least one of the effects including thermal stimulation, thermal shrinkage, coagulation or destruction of the said soft tissue of the eye. The accommodation of the treated eye is enhanced by the change of at least one of the biomechanical property of the eye including the elastic property of the ciliary-body, tissue healing effect, or the available spacing of the ciliary-zonule complex.

Further preferred embodiments of the present invention will become apparent from the description of the invention that follows.

While the apparatus and method has or will be described for the sake of grammatical fluidity with functional explanations, it is to be understood that the claims, unless expressly formulated under 35 USC 112, are not to be construed as necessarily limited in any way by the construction of “means” or “steps” limitations, but are to be accorded the full scope of the meaning and equivalents of the definition provided by the claims under the judicial doctrine of equivalents, and in the case where the claims are expressly formulated under 35 USC 112 are to be accorded full statutory equivalents under 35 USC 112.

### BRIEF DESCRIPTION OF THE DRAWINGS

FIG. 1 is a cross section view of an anterior portion of the eye showing the ciliary body to be treated by the laser procedure in accordance with the present invention.

FIG. 2 is a block diagram of the components of an endoscope-assisted laser system of the preferred embodiment.

FIG. 3 is a schematic diagram of the control panel of the endoscope-assisted laser system of the preferred embodiment.

FIG. 4 is a cross section view of the eye showing the insertion of the endoscope gauge probe which delivers the laser energy to the treated ciliary body.

FIG. 5 shows the plan view of two preferred structures of the gauge probe and the cross section view of the gauge probe having three fibers inside.

FIG. 6 shows the relative position of the angled gauge probe which can cover a treatment angle about 200 degrees for the procedure in accordance with the present invention.

FIG. 7 shows a similar structure of FIG. 6, but having the gauge probe flipped 180 degrees in order to cover the other portion of the treated tissue.

FIG. 8 shows the laser beam position and spot size from the gauge probe end.

FIG. 9 shows the greater detail of the gauge probe and the treated ciliary-body tissue.

FIG. 10 shows one preferred embodiment to treat a pseudo-phakic eye having an IOL implanted.

FIG. 11 shows another preferred embodiment, in which the laser is operated in various modes including cw, even and uneven pulsed mode.

FIG. 12 shows another preferred embodiment, in which the laser is operated in a preheating mode and followed by a pulsed mode.

### DETAILED DESCRIPTION OF THE INVENTION AND THE PREFERRED EMBODIMENTS

### The Mechanisms and Theory

An ophthalmic method and system in accordance with the present invention comprises a laser offering a localized tissue heating of the treated tissue and minimal thermal damage to the non-treated tissue. It also consists of an endoscope device for real time monitoring. The preferred laser spectrum is the region where the treated tissue, the ciliary-body (process) containing blood or melanin has strong absorption, but has high transparency to water, or the vitreous liquid of the eye. Based on these criteria, the preferred laser spectrum includes near infrared (NIR) laser at about (0.7-1.3) micron. Other ranges of spectrum with strong water or protein absorption such as IR laser of (2.8-3.2) microns, or UV laser of (193-300) nm should be excluded. The effects resulted from the laser treatment include thermal stimulation, thermal expansion, thermal shrinkage, coagulation or ablation of the ciliary-body tissue in the anterior chamber of the eye.

We should note that the ‘ablation-type” lasers having wavelength of UV (190-300) nm and NIR (2.8-3.2) microns excluded in the present invention, are required in the prior arts using scleral ablation method. These prior arts include such as U.S. Pat. No. 6,258,082, U.S. Pat. No. 6,263,879, U.S. Pat. No. 6,824,540, U.S. Pat. No. 7,275,545, U.S. Pat. No. 7,867,223, U.S. Pat. No. 8,276,593 and U.S. Pat. No. 8,479,745.

We shall first describe our theory behind the invention for the increase of accommodation amplitude (AA) to treat presbyopia. It has been known that presbyopia was caused by age. However, the complete description for the mechanisms of accommodation is not conclusive, which includes capsular theory (von Helmholtz theory), lens-crowding theory (Schachar theory), Catenary theory (by Coleman, Ophthalmology, 2001, vol. 108, page 1544-51) and the two-component elastic theory by the present inventor (J. T. Lin, “New formulas comparing the accommodation in human lens and intraocular lens” in J. Refractive Surgery, 2005, vol. 21, page 200-201). All those theories, however, have a common principle that ciliary theory (CB) contraction causes lens accommodation for near vision, although lens power increase (or curvature change) may attribute to various mechanisms: via measure gradient between vitreous and aqueous compartments (Coleman, 2001), via relaxation of the lens capsule (Helmoholtz), or via combination of lens relaxation and anterior shift (Lin, 2005). Therefore, the fundamental issue for presbyopia treatment becomes how to improve or enhance the CB contraction which causes the increase of system power or lens power. Both the prior arts of SEB (Schachar U.S. Pat. Nos. 5,489,299, 5,722,952,) and laser scleral ablation (Lin, U.S. Pat. No. 6,258,082, U.S. Pat. No. 6,263,879) are dealing with the superficial layer of the sclera tissue, by sclera expansion or by increasing the elasticity of the laser ablated scleral regions. The change of scleral structure is then translated to the movement (or contraction) of the ciliary body (CB) and the zonular fiber connected to the lens.

Based on the prior arts of Schachar and Lin, the influence from sclera superficial change to CB, then to zonules and lens is rather inefficient due to the ocular structure of the CB-zonule-lens complex and the “remote” distance from the sclera layer to the zonule and lens. In addition, these prior arts are rather invasive surgical procedures taking about (15-30) minutes per eye. The present invention proposes to “directly” change the property of the CB tissue which is closer to the zonules-lens and therefore will be much more efficient. It was reported in a “stretching experiment” (Glasser and Campbell, Vision Research, vol. 38, p. 209-229) that each one mm contraction of CB may induce (0.8 to 2.6) diopter of accommodation in young lens (age 10-53), but almost no power change for old lens (age 54 to 87). This clinical data also supports our theory that lens anterior chamber shift (ACS) or axial movement shall play an important role, particularly in old eyes.

To calculate the total accommodation amplitude (AA), I proposed the “Lin dynamic model” by introducing two components, the anterior shift (A1) or axial movement and lens relaxation (a2) AA=A1+A2=M1(dS)+M2(dR), where lens power change is converted to AA by a factor, CF=(0.7-0.8), rather than 100% conversion. My calculations showed that one mm anterior movement (dS) of the lens will cause about M1=(1.0 to 1.7) diopter of image myopic shift (for patients to see near) and the reversed process, posterior shift will allow the patient to see far. The second component A2 causes the presbyopic lens to see near by lens relaxation with decreased radii of the lens, mainly by the anterior capsule of the lens. For a typical post-surgery patients with an average accommodation amplitude (AA) of +2.0 D, I propose that the AA may attribute to A1 or A2 or their combination, depending on patient age and the lens rigidity.

My numerical calculation shows an increase of AA=2.0 D may be achieved by any of the following: (1) lens relaxation (dR) with decreased radius R1 from 10.56 to 9.0 mm without A1; (2) combining dR with R1 decreased to 9.5 mm (with a lens power change of 1.36 D and system power increase of 1.02 D) and dS of 0.65 mm (with a system power increase of 0.98 D); and (3) an anterior shift (AS) of about 1.33 mm without dR, assuming m1=1.5 (D/mm). Although the lens power change is very sensitive to its radii changes, about (0.8 to 2.7) D/mm as shown by my calculations, its effect on AA however is limited by the rigidity of the lens nucleus or capsule (RLC) and the available amount of ciliary contraction and its spacing to the lens, and the zonular effective length (ZEL) defined by the amount of expansion to allow lens central curvatures (radii) change. The AA given by anterior lens shift (AS) on the other hand is not limited or affected by the condition of RLC, therefore it is still possible to have sufficient AA (say +1.0 D to 1.5 D) by a pure AS without LR, particularly for lens with initial radii smaller than 9.5 mm. noting that AA is inverse proportional to the initial value of (R1, R2).

From the empirical data of Glasser and Campbell (Presbyopia and the optical changes in the human crystalline lens with age. Vis Res. Vol 38:209-229, 1998) and my calculated data, I find that the change of accommodation amplitude (AA) versus ciliary body contraction distance (D) is non-linear in nature. The AA per mm change of D (defined as M), depends on the value of D as follows: M=(1.94, 2.3, 2.6, 1.0, 0.8) D/mm, for D ranges of D=(0.0-0.5), (0.6-1.0), (1.1-1.5), (1.5-2.0) and (2.0-2.5) mm. Based on these data, I estimate that D=(0.4 to 0.7) mm will be needed for a typical AA=(1.8 to 3.5) diopter which is defined as a successful treatment. The prior arts based on superficial sclera ablation (Lin-879) fails to achieve this criteria, particularly after regression. The thermal effects in the present invention with fundamental difference in the mechanisms, location and structure of the treated area, therefore will be more efficient than prior arts.

Furthermore, “pseudo-accommodation” (with major regression) was reported in prior arts of SBE due to globe axial elongation after the treatment, which may also occur in U.S. Pat. No. 6,263,879 of Lin (Lin-879). The superficial scleral expansion (SEB) or ablation (Lin-879) causes the increase of sclera ring radius, but it is very inefficient in affecting the CB contraction. These prior arts also suffer major regression due to sclera healing, as clinically reported.

Clinically, it is important to note that the total accommodation amplitude (AA) is governed by the amount of ciliary body contraction, therefore the AA shall be governed by the tissue property change after the treatment including the elasticity of the sclera or ciliary body (CB) tissue, the available space for CB contraction, or the distance between CB to the lens connected by the zonules fiber. In our proposed “laser induced” thermal shrinkage or expansion (TSE) of the choroids-ciliary-body-zonules complex (CCZ), there is a minimal amount of thermal energy needed in order to cause the TSE of the CCZ soft tissue which include choroids or CB. To cause TSE of the tissue, only those lasers with appropriate spectra can be used, such that the laser energy can be localized absorbed by the treated tissue via the pigments, protein, blood or water content of the tissue.

We note that without the above theoretical analysis, it would be very difficult to predict the clinical outcome. My method in this invention and the parameters for the proposed device and clinical techniques are based upon the above theoretical findings. Further analysis on the mechanisms and efficiency of ciliary body contraction will be discussed as follows.

Using the new method proposed in this invention, we have recently conducted human trials for presbyopia eyes in selected clinics in China, Brazil and Argentina. Patients at age range of 49 to 60 were treated for both eyes having pre-operative presbyopia of 1.5 D to 3.0 D. We observed an mean improvement of about 1.5 D accommodation for treated patients. We also conducted eyes having pre-operative IOP about 60 mmHg which was reduced to less than 25 mmHg on day one after the treatment followed by more improvement in 2 weeks. These initial clinical cases support our proposed theory and the efficacy of the proposed system and method for both glaucoma and presbyopia correction. Specific preferred embodiments will be described as follows.

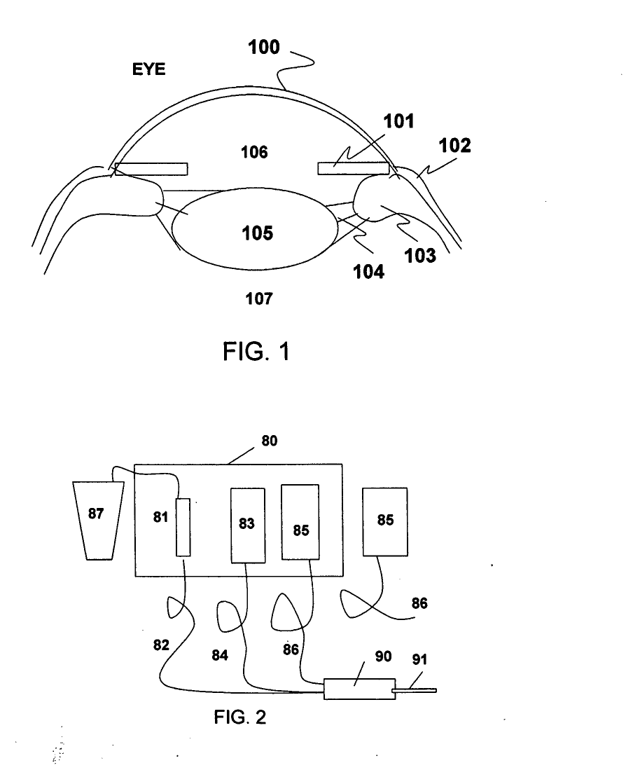

FIG. 1 shows the diagram of a human eye (a side view). The ocular structure of an eye consists of the cornea 100, the iris 101, the sclera 102, and the ciliary-body (CB), or ciliary-process, 103 which is connected to the lens 105 by the zonule 104. The lens shape and its location (or the anterior chamber depth) is governed by the tensile force from the sclera-ciliary-zonule complex and the pressure (or pressure gradient) in the anterior chamber 106 and in the vitreous 107.

From the diagram shown by FIG. 1, I propose the following 2-component mechanisms for efficient CB contraction which causes accommodation. The accommodation amplitude (AA) is given by a 2-component theory AA=A1 (anterior shift)+A2 (lens relaxation). Both A1 and A2 are proportional to the amount of CB contraction which is limited by the elasticity of the sclear-CB-zonule complex (SCZ) and the spacing among each of the SCZ components. Therefore loosening of the SCZ will enhance accommodation The present invention proposes to use laser thermal energy to change the biomechanical property of the SCZ complex, rather than laser ablation of the scleral tissue proposed in prior arts.

### Embodiment—1

One preferred embodiment is to use an endoscope device which allows a real time monitoring of the procedure to be described as follows. As show in FIG. 2, an endoscope-assisted laser system 80 consists of a camera 81 connected to a signal fiber 82, a white light generator 83 connected to an illumination fiber 84 and a laser device 85 connected to a laser fiber 86. These three fibers 82, 84, and 86 are guided to the treating area by an endoscope hand piece 90 having a gauge probe 91 with a diameter about 1.0 to 2.5 mm, which is commercially available and named as gauge No. 19 to 25. The above mentioned three fibers have a core diameter about 0.1 to 0.5 mm, such that all these three fibers can go through the hollow tube of the gauge probe 91. The camera 81 is further connected to a monitor 87 for a real time view of the surgery. The laser device 85 can be integrated inside the box of the endoscope 80 as shown by FIG. 2. It can also be a separate stand along device 85 having its output laser beam delivered by a fiber 86 and coupled to the gauge probe 91. The output laser beam energy from the laser device 85 is delivered, via the gauge probe 91, to the treated ciliary-body (or process) soft tissue 103 of the eye.

### Embodiment—2

This invention proposes a prefer laser device 85 having the output laser beam to be highly transparent to the vitreous liquid of the eye, but strongly absorbed by the pigments of the treated soft tissue, the ciliary body (or process) 103. In addition, visible laser (0.4 to 0.69 nm) should be excluded to avoid potential retina damage. Therefore the preferred laser beam wavelength includes between about 0.7 microns to about 1.3 microns which has very weak absorption in the vitreous liquid. The preferred lasers include solid-state lasers such as Nd:YAG, Ti:sapphire laser having wavelength about (0.7-1.3) microns, or fiber lasers having wavelength about (1.0-1.3) micron, or diode (semiconductor) lasers having wavelength about (0.7-1.3) micron, with the most preferred wavelength about 780 to 980 nm. The preferred laser beam intensity profile includes a continuous-wave (cw) or a pulsed wave having a pulse duration of 1.0 ns to about 500 microseconds. The operation mode of the laser energy delivered to the treated tissue 103 includes a continuous mode or a pulsed-mode having a repetition rate of (0.5-5,000) Hz, where the preferred laser irradiation on and off period includes from about 0.1 ms to about 2 seconds.

### Embodiment—3

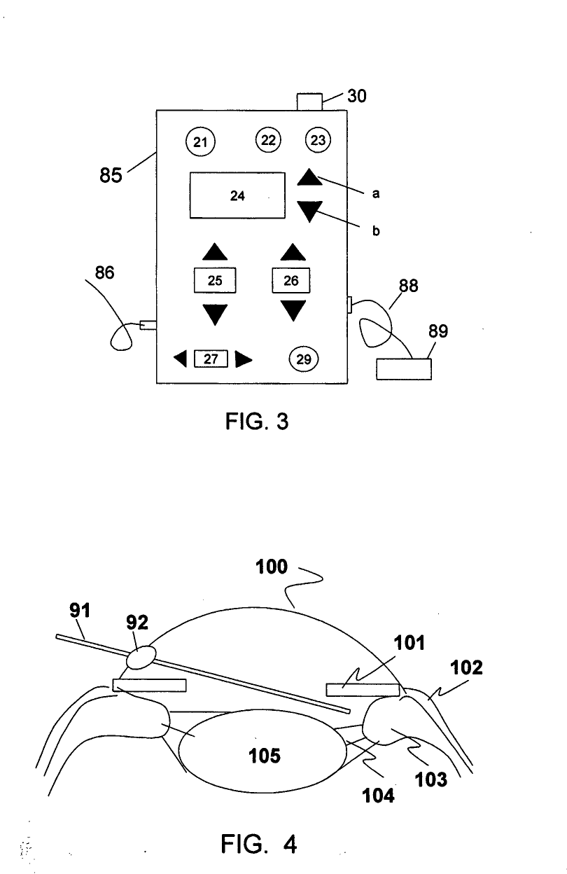

FIG. 3 shows a schematic diagram of the control panel of the endoscope-assisted laser system, where the laser device 85 consists of a laser fiber 86 which is connected to the hand piece 90 and the gauge probe 91 as described in FIG. 2. It also includes an emergent switch 30 and a foot pedal 89 connected to the device by a wire 88, wherein the foot pedal is used to turn on and off the laser power by the surgeon.

The said laser device has a control panel having touch screen control bottoms including an aiming mode 21 (for the aiming red light power adjustment), a ready-mode 22 (for laser turn on and off), a standby mode 23 (for control parameters adjustment mode). It also includes a laser power indicator 24 and level adjustment up (a) and down (b); a laser on-duration indicator 25, a laser off-duration indicator 26; a laser illumination total period (time) indicator 27 (in seconds); and a save-mode 29 to save preset parameters for friendly use. The on-off duration is adjustable between 0.1 ms to 900 seconds and may also be set as a continuous mode, that is the off-duration set at zero and the on-duration set at 900 seconds, or at cw mode. The pulsed train mode with a preheating period and followed by even or uneven pulses may also be set easily.

### Embodiment—4

As shown by FIG. 4, a gauge probe 91 is inserted into the treating area in the anterior chamber in the ciliary sulcus space via an incision 92 on the cornea (size about 1.0 to 2.5 mm), wherein the incision 92 is commonly used in a cataract or phaco procedure. For aphakic eyes having the natural lens 105 removed, there is enough space for the gauge probe 91 to reach the treated tissue 103 without touching the lens 105. However, for phakic eye (with natural lens 105), the lens 105 is required to be pushed down by filling viscoelastic solution in the anterior chamber (underneath the iris 101) to create more pupillary space and allows the gauge probe 91 to reach the treated tissue 103 can be either coagulated or ablated by the laser energy. The treated soft tissue includes the ciliary tissue (or ciliary processes) and its epitheliums, In addition to the combined procedure of presbyopia treatment and cataract, the preferred embodiment also includes the combined presbyopia and glaucoma corrections for old patients having high intraocular pressure (IOP).

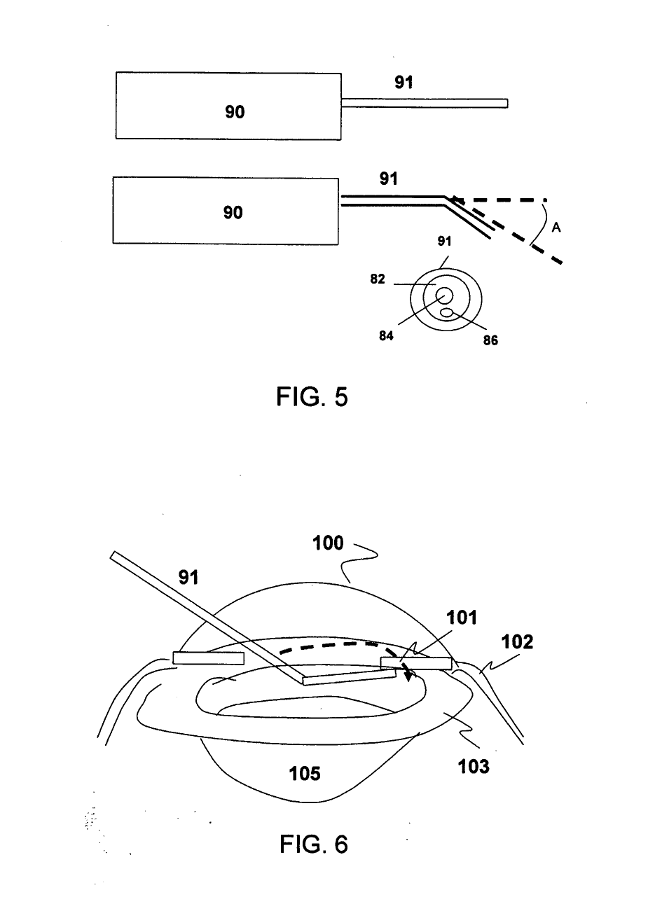

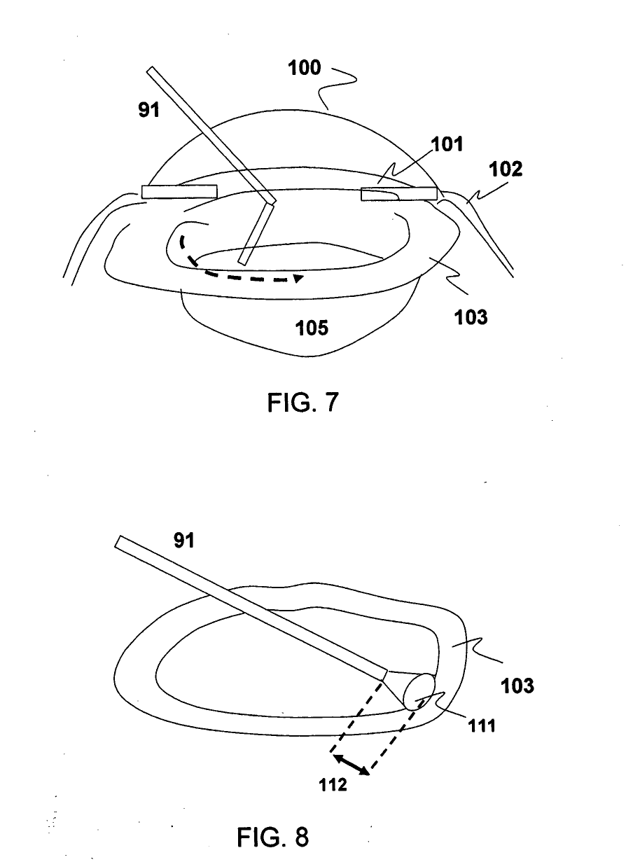

As shown in FIG. 5, The shapes of the endoscope gauge probe 91 includes straight and angled structure having a curved angle of A=20 to 45 degrees. Also shown in FIG. 5 is the cross section of the gauge probe 91 showing the three fibers 82, 84 and 86, where the preferred cross section of the camera-fiber 82 has a larger area than 84 and 86 in order to achieve a better view. For example, in the case of a No. 20 gauge is used, the diameters of each of these fibers include (0.2-0.5) mm for fiber 82 and (0.1-0.2) mm for fiber 84 and 86. A straight gauge probe is easy to be handled but it has a limited treating angle about 200 degree, whereas the angled gauge probe can cover a wider angle up to about 360 degree by flipping the angled-gauge probe as illustrated by FIGS. 6 and 7.

### Embodiment—5

As shown in FIG. 8, the degree of coagulation or ablation of the treated tissue 103 is governed by the temperature increased resulted from the laser beam which has a fluency (F) defined by the laser power (P, in mW or Walt) per unit area, or F=P/(0.25×3.14R̂2), in a unit of (W per cm square, or W/cm̂2); R being the diameter of the laser spot 111 (assuming a round spot) on the surface of the treated tissue 103. The laser beam spot 111 size (R) is further defined by the distance 112 and the beam divergence angle from the end of the laser-fiber inside the gauge probe 91, or the so-called numerical aperture (NA). For R equals 0.2 to 3.0 mm, the preferred parameters are: NA equals 0.2 to about 0.6; laser power on the surface of the treated tissue 103 is 50 mW to about 2,000 mW, or fluency (F) 0.5 W/cm̂2 to about 5.0 W/cm̂2; and the distance 112 is 0.2 to about 3.0 mm. We note that a direct contact of the gauge probe 91 to the surface of the treated tissue 103 should be avoided in order to prevent fiber tip damage or over heating of the treated tissue 103.

### Embodiment—6

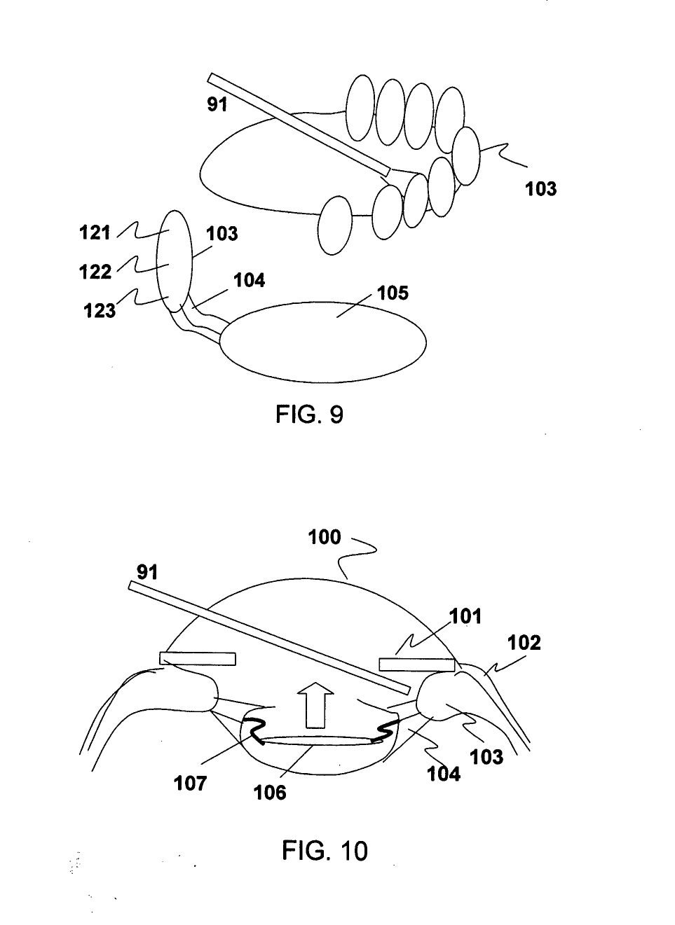

As show in FIG. 9, the operation modes of the treatment include stationary spot to spot illumination on the surface of the treated tissue 103, or a scanning mode over the surface of the treated tissue 103. The number of spots on the surface of the treated tissue 103 includes 15 to 30 spots around an angle of 200 to 360 degrees. The scanning rate includes a rate of 1.0 to 5.0 mm per second. Also shown in FIG. 9 is the three portions 121, 122, 123 of each of the treated ciliary body (process) tissue 103 having its tail portion 123 connected to the lens 106 via the zonule 104. The laser treated area includes one or more than one of the three portions (121, 122, 123), with the most prefer portion 123 which has a direct connection to the lens and hence could cause more efficient reshaping of the lens. The laser-induced shrinkage of the treated ciliary body (process) tissue 103 will result an increasing accommodation of the eye via my two component theory mentioned earlier, namely the lens translation (A1) and the lens reshaping (A2), that is the total accommodation amplitude AA=A1+A2. Based on my published calculations (J. T. Lin, J Refract Surg. 2005; 21: 200-201; and 765-766) an accommodation power (A1) 1.5 to 3.5 D may be achieved for a lens movement of 1.0 to 1.5 mm, whereas A2=0.5 to 0.6 D for each 10% change of the lens front curvature.

From the empirical data of Glasser and Campbell (Vis Res. Vol 38:209-229, 1998), the AA per mm change of ciliary body contraction distance (D) (defined as M), depends on the value of D as follows: M=(1.94, 2.3, 2.6, 1.0, 0.8) D/mm, for D=(0.0-0.5), (0.6-1.0), (1.1-1.5), (1.5-2.0) and (2.0-2.5) mm. Based on these data, I estimate that D=(0.4 to 0.7) mm will be needed for a typical AA=(1.8 to 3.5) diopter which is defined as the criteria of successful treatment. The prior arts based on superficial sclera ablation (Lin-879) fails to achieve this criteria after the post-operation regression. The thermal effects in the present invention has fundamental difference in the mechanisms, location and structure of the treated area and it is more efficient than prior arts.

### Embodiment—7

As shown in FIG. 10, a pseudo-phakic eye (with an implanted intraocular lens, IOL), laser treatment of the ciliary body tissue 103 will enhance the eye accommodation via the IOL translational movement caused by the contraction of the ciliary body 103, where the IOL has two legs 107 which are attached to the lens capsule. The present method provides an alternative for presbyopia treatment without the implant of accommodative IOL (AIOL) which has much higher cost and limited low accommodation power (<2.0 D) comparing to that of the present method, 2.0 D to 4.0 D. In addition to the IOL cases, the present method also apply to cases having cataract surgery, or the aphakic eyes, and the piggy-back cases having an IOL adding to the natural lens. My published calculations (J. T. Lin, J Refract Surg. 2005; 21: 200-201; and 765-766) show that each 1.0 mm of the lens movement will result an accommodation power of 1.5 to 2.0 D, depending on the power of the moving lens. Therefore, one may expects an accommodation power of 1.5 to 4.0 D, for a lens movement of 1.0 to 1.5 mm. We should emphasize that the presbyopia treatment can be combined with glaucoma or cataract procedures to achieve multi-function in single system

### Embodiment—8

To achieve the preferred coagulation temperature of the treated soft tissue 103, about 50 to 65 centigrade, the preferred Illumination time period(t) of the laser on each spot of the treated soft tissue 103 is 0.2 to 5.0 seconds depending on the laser fluency (F) and the laser beam spot size on the surface of the treated tissue 103. The laser induced temperature increase (dT) of the treated soft tissue 103 is proportional to the product of F and t, Therefore a larger F should use a smaller t, or shorter illumination period. For example, t=2 seconds using F=1.0 (W/cm̂2), corresponding to t=1.0 seconds using F=2.0 (W/cm̂2).

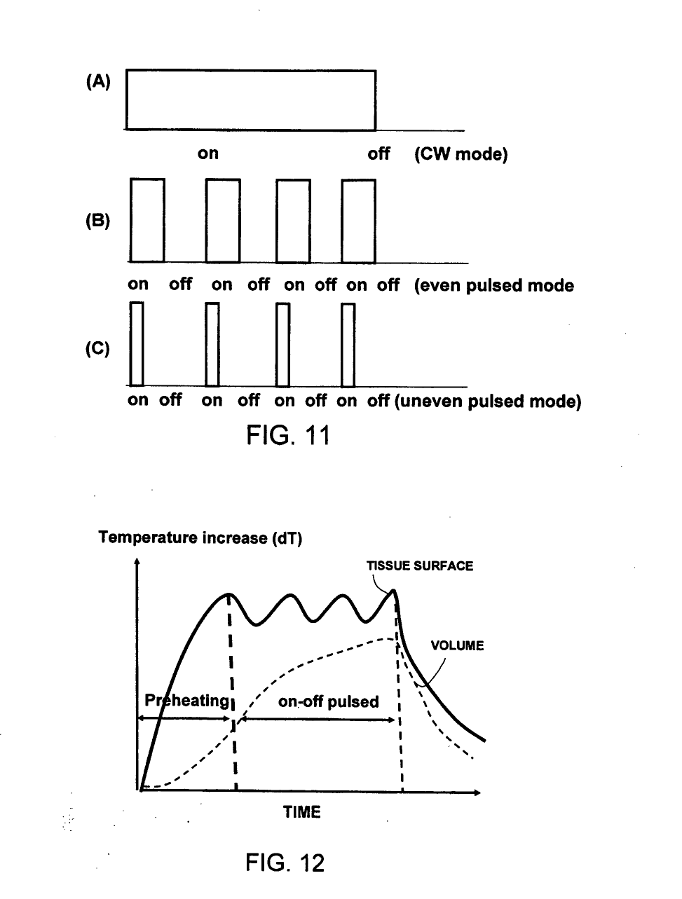

As shown in FIG. 11, the treating mode could be cw (A), or even pulsed mode (B) or uneven pulsed mode (C), where the on and off pulse duration includes 0.1 ms to 2.0 seconds. The preferred pulse duration ratio between the on and off is 0.01 to 1.0, with the most preferred ratio of 0.1 to 0.5, where ratio 1.0 represents the case of even pulsed mode. For deeper laser energy penetration into the treated tissue 103 and more coagulation effects, The present invention also proposes a preheating method.

As shown in FIG. 12, for the temperature increase of the surface of the treated tissue 103, where preheating (for a period of 1.0 to 3.0 seconds) followed by a pulsed mode can offer a deeper volume coagulation, whereas the treated surface temperature (solid curve) is kept at constant and the volume temperature (dashed curve) is increasing gradually. This novel technique was published by the inventor (J. T. Lin, SPIE Newsrooms, paper no. 10.1117/2.1201006.002507, 2010) for laser selective cancer therapy, However, it is the first time presented in this invention for vision correction. We note that the pulsed mode operation described above offers a smaller thermal damage to the surface tissue comparing to the cw mode, whereas it also offers a longer heating period for a maximum volume shrinkage of the treated tissue 103 resulting a higher accommodation effects. We should note that the above described various laser on-off parameters may be achieved by the features described in EMBODIMENT—3 and the schematic diagram of the control panel demonstrated by FIG. 3.

While the invention has been shown and described with reference to the preferred embodiments thereof, it will be understood by those skilled in the art that the foregoing and other changes and variations in form and detail may be made therein without departing from the spirit, scope and teaching of the invention. Accordingly, methods and apparatus, the ophthalmic applications herein disclosed are to be considered merely as illustrative and the invention is to be limited only as set forth in the claims.

I claim:

1. A method for treating presbyopia comprising:
selecting a laser device producing a laser beam having a wavelength from about 0.7 micron to about 1.3 micron with the most preferred wavelength from about 780 nm to about 960 nm;
delivering said laser beam to a predetermined area of the ciliary-body in the anterior chamber of an eye by an endoscope device;
wherein said ciliary-body absorbing said laser beam energy can affect at least one property of an eye such that the accommodation of a presbyopic lens is enhanced,

2. A method of claim 1, wherein said ciliary-body is connected to the lens via the zonule of the eye.

3. A method of claim 1, wherein the thermal energy of said laser beam can result in at least one of the effects including thermal stimulation, thermal shrinkage, coagulation or destruction of the said ciliary-body of the eye.

4. (canceled)

5. (canceled)

6. (canceled)

7. A method of claim 1, wherein the energy of said laser beam is delivered to said ciliary-body at a continuous mode or a pulsed mode; laser exposure duration from about 0.2 seconds to about 5.0 seconds for each spot of said ciliary-body; laser power on the surface of said ciliary-body from about 50 mW to about 2,000 mW; and laser fluence from about 0.5 W/cm̂2 to about 5.0 W/cm̂2.

8. A method of claim 1, wherein the energy of said laser beam is delivered to said ciliary-body by a pulsed mode including an even pulsed mode having equal light-on and light-off duration, a uneven pulsed mode having unequal light-on and light-off duration and a preheating mode followed by a pulsed mode;
wherein the light-on and light-off durations are from about 0.1 mini-seconds to about 2 seconds;
and the preferred ratio between the light-on and light-off duration is 0.01 to 0.1.

9. (canceled)

10. A method of claim 1, wherein said endoscope device further consists of a gauge probe including a straight or angled structure having a curved angle from about 20 to about 45 degrees; the end face of said gauge probe has a distance of 0.2 to 3 mm to the surface of the treated said ciliary-body having from about 15 to 30 treated spots; and said gauge probe can be operated in a stationary or scanning mode covering the treating angle from about 200 to 360 degree in said anterior chamber of the eye.

11. (canceled)

12. A method of claim 1, wherein said presbyopia treatment can increase said accommodation of the lens and also reduce the intraocular pressure of a glaucoma eye to achieve multi-function in single system.

13. A method of claim 1, wherein said laser beam energy is delivered to including one or more than one portion of said ciliary-body with the most preferred portion having a direct connection to the lens of an eye.
